(19) United States
(12) Patent Application Publication
(10) Pub. No.: US 2012/0247482 A1
(43) Pub. Date: Oct. 4, 2012

(54) Adjustable head restraint system

(76) Inventors: Dereck Rizo Patron, Carpinteria, CA (US); Martin Rizo Patron, Carpinteria, CA (US)

(21) Appl. No.: 13/065,933

(22) Filed: Mar. 31, 2011

(51) Int. Cl. A61F 5/00; A61F 5/055

(52) U.S. Cl. 128/845; 602/18

(57) ABSTRACT

A head/occipital support system for emergency response use, intended to be employed in conjunction with a cervical collar. The system achieves very favorable stowabilty while still providing a wide range of height adjustability to accommodate a range of patients with decreased loading on the head/spine alignment. The system also includes at least one strap(s) to secure the patient by bi-laterally connecting each side of the cervical collar to the patient carrier. An accessory wedge is utilizes for patients requiring a wider range of head support.

### RELATED APPLICATIONS

Not Applicable

### FEDERALLY SPONSORED RESEARCH

Not Applicable

### SEQUENCE LISTING

Not Applicable

### BACKGROUND OF THE INVENTION

The invention relates to support of the head or occipital region of patients with possible spinal trauma and in particular to a support system particularly suitable to the needs of emergency responders.

Emergency response procedures for patients with suspected-spinal trauma usually involve immobilizing the patient on to a carrier device, such as a backboard, gurney, rescue basket, stretcher, litter and the like, in a manner that is intended to maintain neutral alignment of the head and spine. Ideally, no further twisting or displacing of the head, neck and spine are done until the patient is delivered to a care facility. Thus the goal of spinal trauma procedure is to restrain the patient so that alignment is preserved and no pressure is put on the spinal/head region during initial restraint and transport of the patient. Unfortunately, in practice it is difficult to accomplish this, in large part due to the configuration of the restraint and support tools currently available to emergency responders.

Often a cervical collar is put on the patient to maintain head/neck/spine alignment. After a cervical collar is in place, most procedures call for the patient's head and torso to be restrained on a carrier. Typically, some type of head/occipital support is placed under the patient and the patient is strapped or taped in place, usually with at least one restraint over the head (or cervical collar). Due to the limitations involved with current emergency response tools, this process has several serious drawbacks.

Current emergency response head supports are designed to restrain the head from moving laterally, vertically, inline and from rotating. Currently available supports are typically some sort of block or fixed wedge designed to secure the head firmly when strapped down. They offer little or no height adjustment. This is a problem because when the head is strapped down, even with a cervical collar, strain is applied because bodies of different sizes and/or spine/neck shape will experience different amounts of loading and dynamic forces during packaging and transport unless they happen to fit the support structure exactly. Basically most patients will either experience undesirable upward or downward leverage of their head relative to their spine when the straps are tightened (Secured/Attached/fastened) due to the lack of adjustability of the height of the occipital/head support needed to keep the head in alignment as well as unwanted forces that can produce distraction on the cervical spinal region due to the movement of the torso while the head has been completely secured to the carrying device. Moreover many of the currently available supports are bulky, with dimensions on the order of several inches in thickness, height and length. Space in emergency response vehicles is at a premium, so storage of such large devices is problematic, and often limits the number of devices that a rescue team can carry, therefore minimizing the number of patients that can be provided with timely treatment. And in fact in many cases multiple response teams may have to respond to the scene of an accident for this reason, when it would be so much better to provide adequate timely treatment as soon as possible.

In addition, the direct strapping of the head is extremely stiff longitudinally. This is a drawback when the patient is carried up and down ramps or stairs, or when the emergency vehicle experiences sudden accelerations or decelerations or other vehicle motions. Both loading due to gravity or movement of the body can cause further strain, distraction and potentially further damage at the site of the injury if the head is too rigidly attached to the carrier. Also, some of the larger, bulkier head restraints use straps for securing the patient, but these units are expensive and not disposable requiring decontamination after each and every use. Other more compact, disposable units typically require adhesive tape as a restraint, which can fail to adhere to wet patient carrying equipment, stick to gloves as well as pick up dirt or debris, often further complicating an already difficult situation for emergency responders. Thus it is the object of this invention to provide a support system that is space efficient when stowed, provides for a large degree of height adjustment, secures the patient for vertical and lateral movement only in a manner that reduces the patient's unwanted manipulation due to loading forces created during transport, is disposable, inexpensive and doesn't involve require the use of adhesive tape.

### BRIEF SUMMARY OF THE INVENTION

The invention includes a head/occipital support adapted to store as a relatively thin, flat piece consisting of sections which may be stacked during use to provide variable height depending on number of sections stacked. The sections may be stacked in a variety of ways, including breaking the support into sections along perforations, folding the section along foldlines, or removing the sections attached, such as by Velcro® or adhesive, to a backing. Preferably the sections include adhesive, Velcro® or the like to facilitate attachment to themselves and to the carrier

The invention also includes at least one adjustable securing strap, the strap(s) adapted to connect at least two opposing regions of the carrier to attachment points on each side of the patient's head. The strap(s) are adjustable. In one version, the carrier end may be a Velcro® closable loop intended to attach to the openings commonly found on the sides of backboards or litters, and adjustable by the closure length of the loop. The attachment to the cervical collar may also be by Velcro®, or by buckles including adjustable strap retainers

In preferred embodiments, the sections are substantially rectangular or square, with possible cut-outs for storage and handling. Preferable dimensions range from sections being on the order of a few inches in length and width, and around a half inch thick. Three to four sections per support are preferred and the total length should be less than typical carrier width, about 18 inches. The material should be moisture resistant, stiff enough to provide support but still flexible, such as common closed cell foams. The perforated version is also preferred, with adhesive strips covered by removable film disposed on the top of over the adhesive and on the bottom of the sections. Such a support is sufficiently stowable that it can be included with an emergency type cervical collar at little or no space penalty

For patients where the planar, sectionable support does not provide enough height adjustment, the invention includes a novel wedge support as an accessory, including two wedge shaped pieces with corresponding, fingers and slots cut into the angled wedge section. These wedges are disposed such that the fingers/slots are interlaced when the pieces are placed together. They may be fully interlaced for storage and the two pieces then slid apart during use to provide a height adjustable v-shaped support. Relatively few of these wedge accessories are need to be carried by the response vehicle as the planar, sectionable support will accommodate most patients.

### BRIEF DESCRIPTION OF THE DRAWINGS

The invention will be better understood by referring to the following figures.

FIG. 1 illustrates the basic features of the novel support.

FIG. 2 illustrates the stacking of sections of the support.

FIG. 3 illustrates one embodiment for fixing the support to the carrier.

FIG. 4 illustrates the novel system including straps adapted to attach to a cervical collar, and one embodiment for strap attachment to the carrier.

FIG. 5 illustrates one embodiment for attaching the straps to the collar.

FIG. 6 illustrates an important advantage of the novel system.

FIG. 7 illustrates an optional wedge support for cases where more height adjustment is required.

FIG. 8 illustrate the use of the novel wedge support.

### DETAILED DESCRIPTION OF THE INVENTION

The invention is a novel sectional head support for use in patient head restraint on a patient carrier, such as a backboard, gurney, rescue basket, stretcher, litter and the like, for emergency response applications. The support is highly stowable while still providing, during deployment, a wide degree of head to carrier height adjustment to minimize loading and unwanted movement on the head-spine axis for a range of patient size and body types. The support may be made out of inexpensive material such as closed cell foam, so it is disposable and inexpensive. The novel support is used with securing straps, rather than directly taping the head as in current disposable systems. In the novel system, the straps attach the patient carrier to attachment points on each side of the head. In the preferred embodiment, the attachment points are on each side of the cervical collar placed on the patient. Thus the invention preferably requires that the cervical collar used with the invention include means for strap attachment. However, other accessories which provide attachment points on each side of the head are conceivable and fall within the broad interpretation of the invention. A suitable collar is the X-Collar, produced by Emegear, the assignee of the current invention.

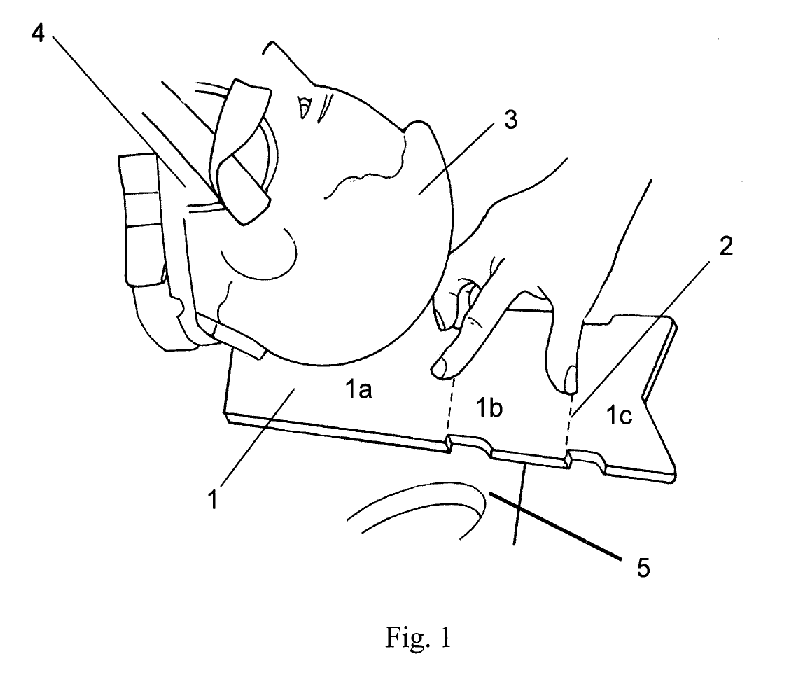

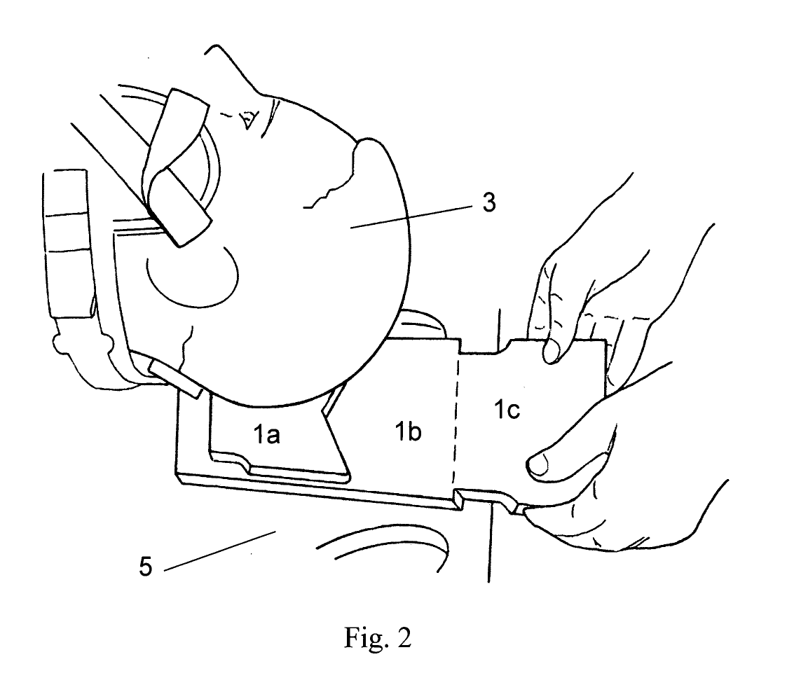

The stowable support portion of the invention is illustrated in FIG. 1. The support 1 is preferably a substantially planar structure, consisting of multiple sections. In the exemplary case shown in the figure, three sections, 1a, 1b, and 1c are shown, but within reason, other numbers such as 4 or more may be used. Two sections are possible but would be limited in adjustability. The sections are separable and stackable. A variety of separation approaches are possible. One preferred approach is to perforate support 1 along separation boundaries 2 allowing for sections to be broken off as needed. The support is placed between head 3, with cervical collar 4 in place, and patient carrier 5. As shown in FIG. 2, section 1a has been broken off and stacked on support 1. Thus depending on how many sections are broken off and stacked, the gap between head 3 and carrier 5 may be filled for a variety of patient size and body shapes with out adding strain to the spine/neck and maintaining proper cervical spine alignment.

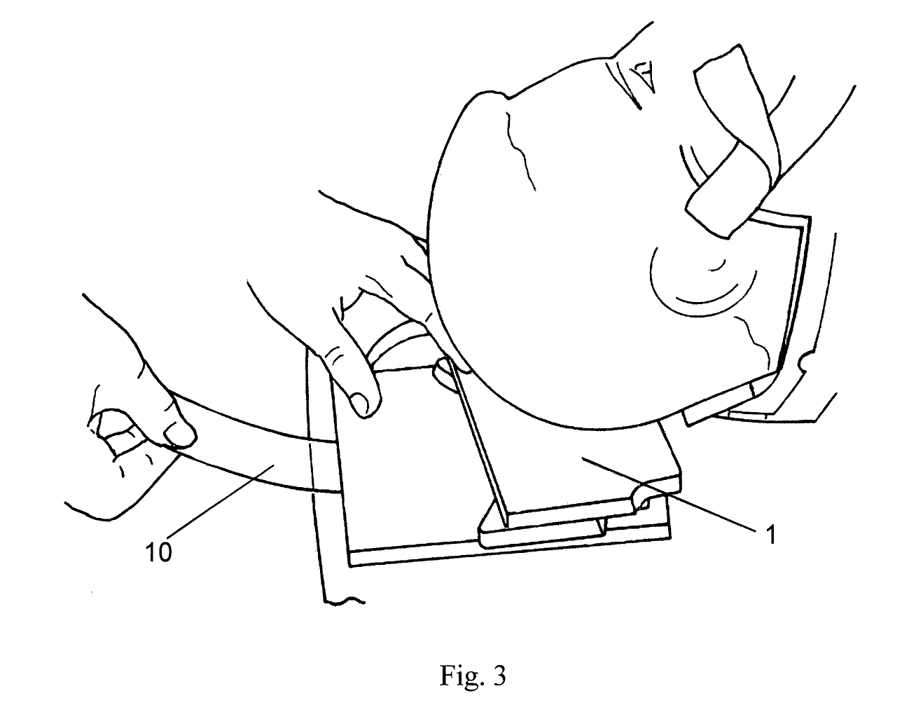

In stowable form, the support is a flat piece, easily stacked or packaged with a cervical collar, for very convenient and space efficient stowage in an emergency vehicle. Yet despite the space efficiency, the support still has far more patient height adjustability then any currently available emergency response head restraint support. Obviously many variations on the shape and section arrangement are possible. For instance instead of perforated section boundaries, fold lines may be employed to stack the sections by folding. Another possible arrangement is the sections may be precut. In preferred embodiments the support is basically rectangular, with possible handling cutouts. Although specific dimensions obviously may vary, the sections should be at least on the order of a few inches on a side to provide adequate area for occipital support. The overall length of the support before sectioning should fit well within the width of carriers, so should be preferably be no more then 12-18 inches. Thickness is a trade-off between stowability and height adjustment range. The inventors have found that a four section piece, a half inch thick provides a one half to two inch range, with half inch increments. Thus is adequate for most patient body types. The material should be inexpensive, moisture resistant, firm enough to provide support while flexible enough for comfort and to allow for folding or breaking. Many materials fit these requirements such as common closed cell foams. Also as shown in FIG. 3, to facilitate placement and to hold the support in place, adhesive with removable cover 10 may be placed on the bottom of the support 1 both to fix to the carrier and between stacked sections.

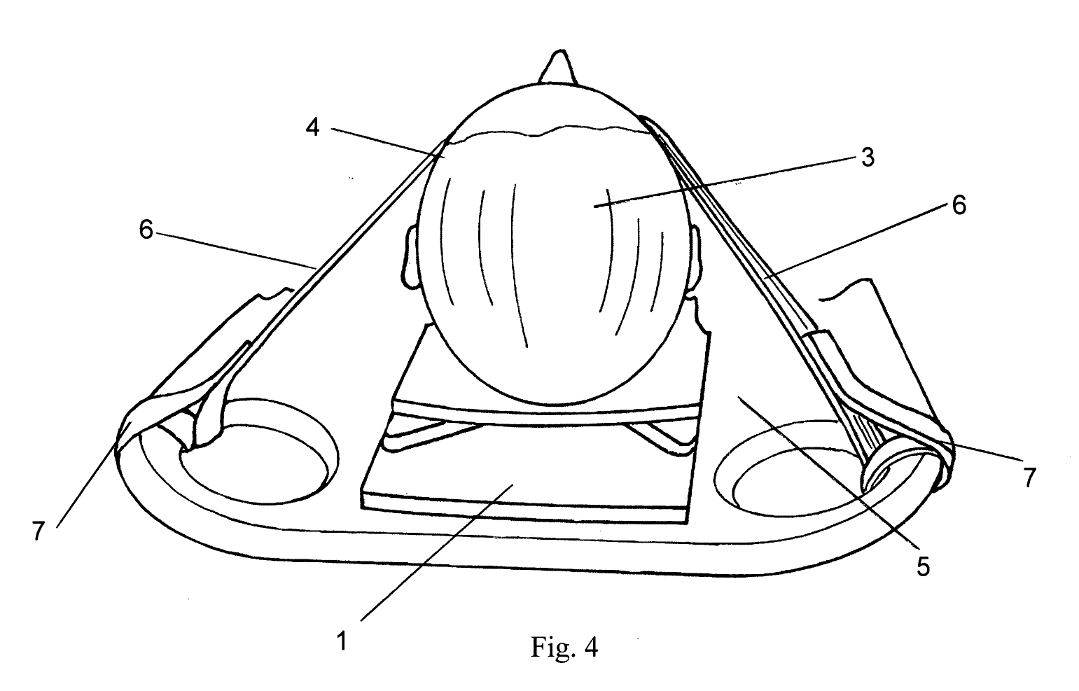

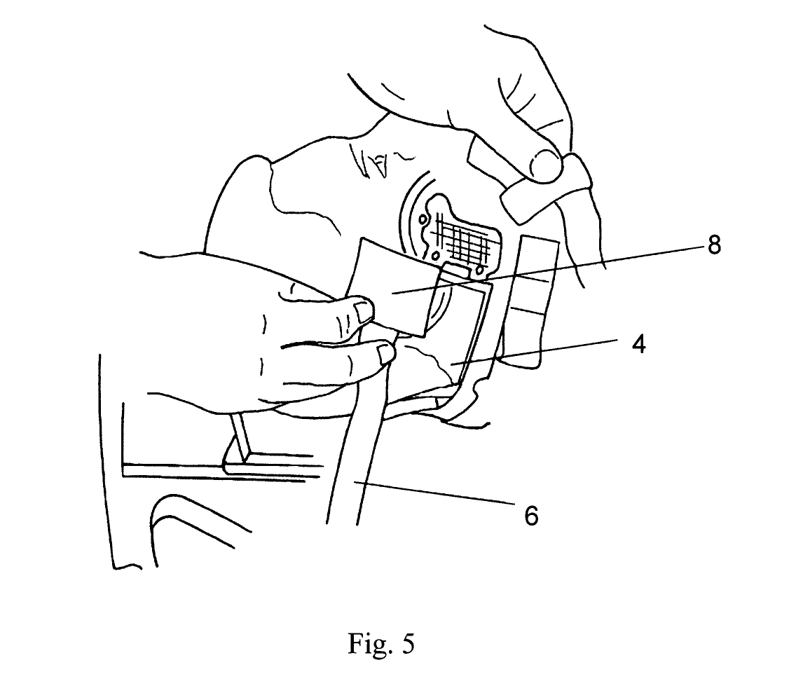

The invention also includes novel provision for securing the head using head attachment means, preferably attachment points on a cervical collar, to facilitate a superior strapping arrangement compared to current emergency support systems. Referring to FIG. 4, two securing straps 6, one on each side of the head 3, supported by adjustable support 1, connect the carrier to the cervical collar 4 as shown. This bi-lateral arrangement both stabilizes the collar laterally as well as vertically onto the carrier. Straps need to be adjustable to provide some tension and readjustment as needed. A variety of attachment/adjustment means are possible. Since many patient carriers 5, such as backboards and litters have openings along the sides, a preferred attachment 7 is a Velcro® closable loop, which may be looped through the opening, and the slack taken up by where the loop is closed. Other fastening arrangements within the scope of the invention will suggest themselves to skilled practitioners. As shown in FIG. 5, the straps 6 also attach on each side of collar 4. A preferred simple solution is shown at 8 where a Velcro® pad on strap 6 attaches to an existing Velcro® pad on collar 4. Locking buckles are also possible, and these could include adjustable strap retainers. Although two straps are shown in the Figure, one skilled in the art will readily appreciate that the use of two straps is not required. The goal is to connect a strap attachment point on one side of a patient's head with a strap attachment point on the same side of the carrier and to do the same thing on the other side of the head/carrier. This could be done with one strap, for example, that had provision for attachment to the head attachment point with a service loop in between that went over or around the head. As long as the slack could be taken up at the carrier attachment points, such an arrangement would be equivalent to the two strap example in the figure. Alternatively more than one strap on each side could also be employed. Thus the two straps shown in the figures are by way of example only.

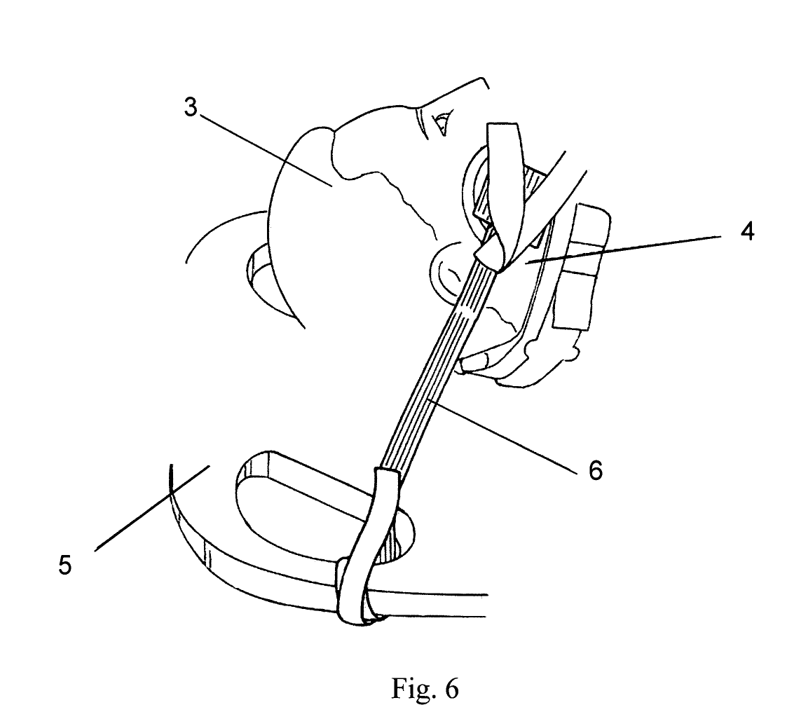

A critical advantage of the novel support system is shown in FIG. 6. Securing Straps 6 are connected between carrier 5 and collar 4 as a relatively long arm with endpoints capable of a degree of pivoting. This arrangement has the effect that although it stabilizes the head laterally and vertically, it is not particularly stiff along the body axis. In Emergency situations, once the patient is secured to the carrier, during movement up and down ramps or stairs, in and out of the vehicle, and during vehicle motion, significant loads can be placed along the body axis. The strap attachment scheme clearly shown in the figure allows for some compliance along this axis, resulting in avoiding further trauma to the spine/neck when exposed to loads along the body axis than compared to existing systems.

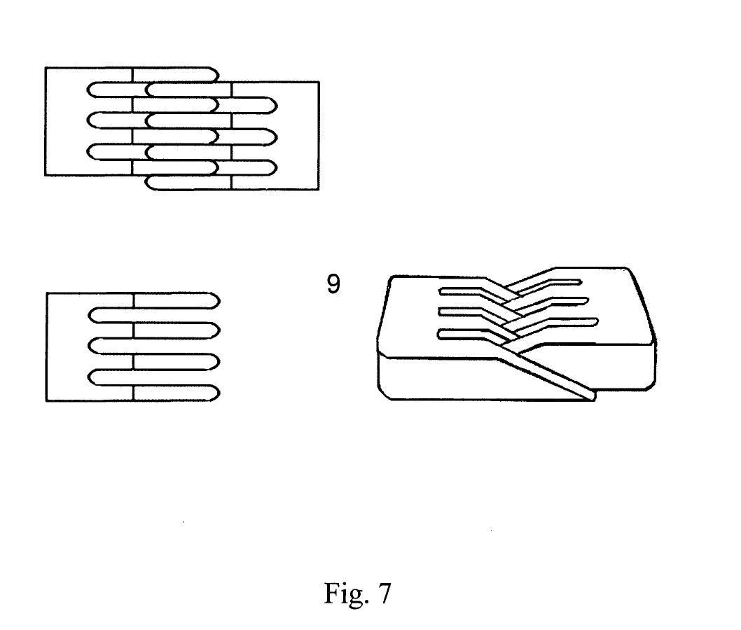

As clearly shown, the novel support and strap system is inexpensive and disposable, space efficient during stowage, has a great degree of height adjustability, and provides a superior, less traumatic securing arrangement. In the preferred approach the support/straps are packaged with the collar, ensuring all of the needed tools are available to the responder in a convenient manner. However, even with the wide range of adjustability provided, some patients will fall outside the range covered by the support. Thus the invention optionally includes another item, which may be carried in smaller numbers for the out of range cases. This item is shown in FIG. 7. It is basically a wedge support 9 consisting of two pieces with slots and fingers cut into an angled surface as shown. The slots and fingers are sized to allow for interlacing as shown. Thus in storage, the pieces are pushed all the way together forming a compact block. During use they may be pulled apart to provide the desired height of support. The V-shape is advantageous for head support as well. Again adhesive with removable covering or Velcro® may be used to facilitate placement of the support. Such a wedge has several advantages over other wedge block configurations. For a suspended object, such as a head held at the proper alignment, the two interlaced wedges may be slid into place to the desired height with no forces applied to the suspended object of any kind, yet still providing lateral and vertical support once placed. Such a wedge may have application in many areas beyond head support for emergency response.

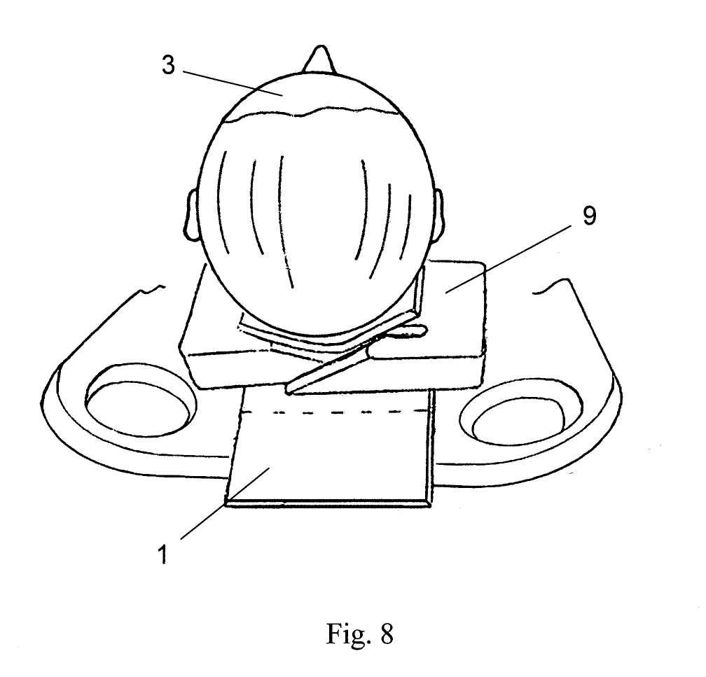

Use of the wedge is illustrated in FIG. 8. The planar support 1 is shown with the wedge 9 suitably placed to support head 3 in alignment with, the spine for a patient with a large vertical alignment gap between the head and spine.

It will be apparent to the skilled artisan that there are numerous changes that may be made in embodiments described herein without departing from the spirit and scope of the invention. Other features not mentioned in the specification, but known to one skilled in the art may be integrated as well without departing from the spirit and scope of the present invention. There are, for example, a wide array of materials, apparatuses, and methods which may be interchangeably used and there are many changes that may be made in dimensions and so forth to accommodate different needs which may be used, all within the scope of the invention. The methods, system, and apparatuses of the present invention should therefore be afforded the broadest possible scope under examination. As such, the invention taught herein by specific examples is limited only by the scope of the claims that follow.

1. A head or occipital support system for use with a patient carrier such as a backboard, gurney, rescue basket, stretcher, or litter, comprising;
A multi section support, comprising at least two stackable sections adapted to store with the sections substantially flat and co-planar and stackable in use on the carrier to a height variable by the number of sections stacked; and,
at least one adjustable securing strap, the strap(s) adapted to connect at least two opposing regions of the carrier to attachment points on each side of the patient's head.

2. The multi section support of claim 1 comprised of non-absorbent material.

3. The multi section support of claim 2 wherein the non-absorbent material is a closed cell foam.

4. The multi section support of claim 1 wherein the sections are stackable by at least one of;
separating the sections along a perforable line for stacking, folding along a fold line for stacking or,
stacking precut sections.

5. The multi section support of claim 1 wherein the sections are on the order of a few inches on a side, approximately half inch thick, and the full support is less than 18 inches in length.

6. The multi-section support of claim 1 further comprising adhesive backing with removable cover disposed to facilitate stacking and secure placement on the carrier.

7. The strap(s) of claim 1 comprising a closable Velcro® loop adapted to loop through an opening in the carrier and close for attachment, wherein the attachment point determines tightness of the strap.

8. The system of claim 1 wherein the attachment points on the patient's head are on each side of a cervical collar.

9. The straps of claim 8 comprising a Velcro® pad adapted to attach to a corresponding pad on the cervical collar.

10. The strap(s) of claim 8 comprising a buckle adapted to lock to a corresponding receiver on the cervical collar.

11. The strap(s) of claim 10 wherein the buckle includes a pull-tension strap retainer.

12. A wedge support, comprising;
two wedge shaped pieces, with corresponding interlacing fingers and slots cut into the angled surfaces of the wedge pieces, wherein in storage the fingers and slots may be fully interlaced to minimize storage space, and when in use the wedges may be pulled apart to a desired support height.

13. The wedge support of claim 12 comprising
adhesive backing with removable cover on the bottom of the wedge pieces to facilitate placement on a surface.

14. A head or occipital support system for use with cervical collar and a patient carrier such as a backboard, gurney, rescue basket, stretcher, comprising;
a system carried for each cervical collar, comprising;
a multi section support, comprising at least two stackable sections adapted to store with the sections substantially flat and co-planar and stackable in use on the carrier to a height variable by the number of sections stacked; and,
at least one adjustable securing strap, the strap(s) adapted to connect at least two opposing regions of the carrier to attachment points on each side of the patient's head; and,
an accessory comprising two wedge shaped pieces, with corresponding interlacing fingers and slots cut into the angled surfaces of the wedge pieces, wherein in storage the fingers and slots may be fully interlaced to minimize storage space, and when in use the wedges may be pulled apart to a desired support height
